(19) United States
(12) Patent Application Publication
(10) Pub. No.: US 2009/0170823 A1
(43) Pub. Date: Jul. 2, 2009

(54) Dosage Regimens and Pharmaceutical Compositions and Packages for Emergency Contraception

(75) Inventor: Charles E. DILIBERTI, Montclair, NJ (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Duramed Pharmaceuticals, Inc., Cincinnati, OH (US)

(21) Appl. No.: 12/339,329

(22) Filed: Dec. 19, 2008

Related U.S. Application Data
(60) Provisional application No. 61/008,287, filed on Dec. 20, 2007.

(51) Int. Cl. A61K 31/56; A61K 31/536

(52) U.S. Cl. 514/171; 514/230.5

(57) ABSTRACT

The present invention is directed to dosage regimens for emergency contraception using nonsteroidal progestins and pharmaceutical compositions and packages thereof. Such regimens are useful for females in need of emergency contraception.

This application claims the benefit of the filing date of U.S. Application No. 61/008,287, filed Dec. 20, 2007, the contents of which are hereby incorporated by reference in its entirety.

### BACKGROUND OF THE INVENTION

1. Field of the Invention

The present invention is directed to dosage regimens for emergency contraception using nonsteroidal progestins and pharmaceutical compositions and packages thereof. Such regimens are useful for females in need of emergency contraception.

2. Background Art

Emergency contraception is generally understood to mean the application of contraceptive measures to a female after an act of sexual intercourse (postcoitus) or undesired insemination, especially after unprotected sexual intercourse. Emergency contraceptive pills (ECPs) and intrauterine devices (IUDs) are the currently available forms of emergency contraception. These methods act both to prevent ovulation or fertilization and possibly post-fertilization implantation of a blastocyst (embryo).

Currently available ECPs, also known as emergency contraceptives (EC), contain higher doses of the same steroidal compounds (estrogens and progestins, or progestins alone) found in regular or conventional daily oral contraceptive pills. The progestin-only method uses levonorgestrel (a synthetic progestogen) in two doses of 0.75 mg 12 hours apart (e.g., Plan B®, Duramed Pharmaceuticals, Inc., Montvale, N.J.) or in a single dose of 1.5 mg within 72 hours of coitus. The combined or Yuzpe regimen uses both ethinyl-estradiol (0.1 mg) and levonorgestrel (0.5 mg) in two doses 12 hours apart within 72 hours of coitus. The mifepristone method uses a large dose of mifepristone, an antiprogestin, either as an ECP or as an abortifacient, depending on whether it is used pre- or post implantation. Emergency contraceptive methods are described in Von Hertzen, H. et al., Lancet, 352:428-432 (1988); Ho, P. C. et al., Human Reproduction, 8(3):389-392 (1993); U.S. Appl. Pub. No. 2005/0032755; WO 2007/000056; and Von Hertzen, H. et al., Lancet, 360:1803-1810 (2002). Additionally, off-label use of high dose(s) of conventional combined or progestin-only oral contraceptive pills are also available for emergency contraception.

Females using these methods of emergency contraception can suffer from one or more steroid-related side effects, including nausea, vomiting, dizziness, fatigue, headache, breast tenderness, lower abdominal pain, diarrhea, and some irregular bleeding or spotting.

### BRIEF SUMMARY OF THE INVENTION

The present invention is directed to a method for emergency contraception, comprising administering to a female in need thereof a dosage comprising a nonsteroidal progestin equivalent to about 0.5 mg to about 20 mg of tanaproget, wherein the dosage is effective for emergency contraception.

In some embodiments, the nonsteroidal progestin is administered within about 72 hours postcoitus.

In some embodiments, the nonsteroidal progestin is administered orally.

In some embodiments, the nonsteroidal progestin is tanaproget. In some embodiments, the tanaproget is in an amount of about 1 mg to about 20 mg. In some embodiments, the tanaproget is in an amount of about 3 mg to about 10 mg.

In some embodiments, the dosage of the nonsteroidal progestin effective for emergency contraception can further comprise an estrogen equivalent to about 0.025 mg to about 0.4 mg of ethinyl estradiol. In some embodiments, the estrogen is ethinyl estradiol.

In some embodiments, the method can further comprise administering a second dosage of a nonsteroidal progestin equivalent to about 0.5 mg to about 10 mg of tanaproget to the female in need thereof.

In some embodiments, the second dosage is administered to the female in need thereof within about 36 hours after administration of a first dosage. In other embodiments, the second dosage is administered to the female in need thereof within about 12 hours after administration of a first dosage.

In some embodiments, the nonsteroidal progestin of the second dosage is tanaproget. In some embodiments, the tanaproget in each of the first and the second dosages is in an amount of about 0.5 mg to about 10 mg. In other embodiments, the tanaproget in each of the first and the second dosages is in an amount of about 1.5 mg to about 5 mg.

In some embodiments, the first and/or the second dosage further comprises an estrogen equivalent to about 0.025 mg to about 0.4 mg of ethinyl estradiol. In some embodiments, the estrogen is ethinyl estradiol.

The present invention is also directed to a method for emergency contraception, comprising administering within about 96 hours postcoitus to a female in need thereof a pharmaceutically effective amount of a nonsteroidal progestin for emergency contraception.

In some embodiments, the nonsteroidal progestin can be administered within about 72 hours postcoitus, within about 48 hours postcoitus, or within about 24 hours postcoitus.

In some embodiments, the method can further comprise administering a pharmaceutically effective amount of an estrogen substantially concurrent with the nonsteroidal progestin. In some embodiments, the estrogen is ethinyl estradiol.

In some embodiments, the invention is directed to a method for on-demand contraception, comprising administering to a female in need thereof a dosage comprising a nonsteroidal progestin equivalent to about 0.5 mg to about 20 mg of tanaproget, wherein the dosage is effective for on-demand contraception. In some embodiments, the dosage is administered to a woman about 6 hours precoitus.

The present invention is also directed to a pharmaceutical package for emergency contraception, the package comprising: (a) a dosage comprising nonsteroidal progestin in a pharmaceutically effective amount for emergency contraception; (b) a suitable container; and (c) a label directing administering the nonsteroidal progestin to a female need thereof within about 96 hours postcoitus.

In some embodiments, the nonsteroidal progestin in such pharmaceutical package is tanaproget.

In some embodiments, the pharmaceutically effective amount of the tanaproget in such pharmaceutical package is about 1 mg to about 20 mg. In other embodiments, the pharmaceutically effective amount of tanaproget in such pharmaceutical package is about 3 mg to about 10 mg.

In some embodiments, the dosage in the methods and packages of the invention is in the form of a capsule or a tablet.

In some embodiments, the label of the pharmaceutical package directs administering the nonsteroidal progestin within about 72 hours postcoitus to a female in need thereof.

In some embodiments, the package can comprise two dosages comprising a nonsteroidal progestin.

In some embodiments, the pharmaceutical package further comprises a dosage comprising an estrogen equivalent to about 0.025 mg to about 0.4 mg of ethinyl estradiol. In some embodiments, the estrogen in the package is ethinyl estradiol.

The invention is also directed to a pharmaceutical composition comprising a nonsteroidal progestin in a pharmaceutically effective amount for emergency contraception. In some embodiments, the nonsteroidal progestin is tanaproget. In some embodiments, the tanaproget can be in an amount of about 1 mg to about 20 mg or about 3 mg to about 10 mg. In some embodiments, the composition is in a form of a capsule or a tablet. The composition can further comprise an estrogen equivalent to about 0.025 mg to about 0.4 mg of ethinyl estradiol. In some embodiments, the estrogen is ethinyl estradiol.

### DETAILED DESCRIPTION OF THE INVENTION

The present invention is directed to methods for emergency contraception. The method comprises administering, within a period of time appropriate for postcoitus emergency contraception, a pharmaceutically effective amount of a nonsteroidal progestin to a female in need thereof, and optionally an estrogen. Nonsteroidal progestin can provide an emergency contraceptive effect, but with less risk of adverse effects in comparison to other steroidal progestins.

The present invention is also directed to pharmaceutical packages for emergency contraception.

### Methods for Emergency Contraception and Compositions Thereof

The present invention is directed to a method for emergency contraception, comprising administering to a female in need thereof a dosage comprising a nonsteroidal progestin equivalent to about 0.5 mg to about 20 mg of tanaproget effective for emergency contraception.

The present invention is also directed to a method for on-demand contraception, comprising administering to a female in need thereof a dosage comprising a nonsteroidal progestin equivalent to about 0.5 mg to about 20 mg of tanaproget effective for on-demand contraception.

The term “emergency contraception” refers to the application of a contraceptive measure post coitus or undesired insemination to prevent pregnancy. In some embodiments, the invention of the present invention is directed to “on-demand” contraception. The term “on-demand contraception” refers to the application of a contraceptive measure less than 12 hours before coitus to prevent pregnancy, i.e., precoital administration of a single dosage of the nonsteroidal progestin equivalent to a woman in need thereof. Examples of on-demand contraception can be found, e.g., in U.S. Appl. Pub. No. 2007/0111976.

Without being bound by theory, in some embodiments emergency contraception (and also on-demand contraception) is achieved primarily by inhibiting or delaying ovulation, decreasing the cervical mucosa, prevention of fertilization, impairment of the transfer of egg or gamate, alteration of the cervical mucous, and/or inhibition of implantation. In some embodiments, emergency contraception is achieved primarily by inhibiting or delaying ovulation, and/or decreasing the cervical mucosa.

The present invention is also directed to a method for emergency contraception, comprising administering within about 96 hours postcoitus to a female in need thereof a pharmaceutically effective amount of a nonsteroidal progestin for emergency contraception. In some embodiments, a pharmaceutically effective amount of a nonsteroidal progestin is administered within about 72 hours, within about 48 hours, or within about 24 hours postcoitus to a female in need of emergency contraception.

The present invention is also directed to a method for on-demand contraception, comprising administering less than 12 hours before coitus to a female in need thereof a pharmaceutically effective amount of a nonsteroidal progestin for on-demand contraception. In some embodiments, a pharmaceutically effective amount of a nonsteroidal progestin is administered less than 8 hours, less than 6 hours, or less than 2 hours before coitus to a female in need of on-demand contraception.

The invention is also directed to pharmaceutical compositions comprising a nonsteroidal progestin in a pharmaceutically effective amount for emergency contraception.

As used herein, “administering” refers to placing or delivering a pharmaceutically effective amount of the desired active agent, for example, a nonsteroidal progestin or an estrogen, so that the active agent(s) is in physical contact with the body of the female in need of such agent. Examples of such administration include providing the desired active agent by routes, such as, but not limited to, parenterally, subcutaneously, intravenously, intramuscularly, transdermally, buccally, orally, or intravaginally.

As used herein, “female” refers to any animal capable of conception. As such, female includes human and non-human mammals, such as, but not limited to, female domestic and farm animals, zoo animals, sports animals, and pets. In some embodiments, the term female refers to a human female. The human female can be of any age. For example, in some embodiments, the female is a pubertal human, i.e., a female between about 11 years old and 17 years old; or an adult female, i.e., a female of about 18 years old or older. In some embodiments, the female is pre-menopausal, or pre-perimenopausal.

The term “pharmaceutically effective amount” of a nonsteroidal progestin refers to a dosage that provides emergency contraception. Such a dosage is one that is sufficient as a single dose or in multiple doses to impart a contraceptive effect to the female to whom it is administered. In some embodiments, such dosage does not cause excessive toxicity, irritation, allergic response, or other possible complications commensurate with a reasonable benefit/risk ratio.

As used herein, the term “about,” when used in conjunction with a numerical amount, means plus or minus 10% of that numerical amount. For example, the term “about 20 mg” would encompass 20 mg plus or minus 2 mg.

Progestins are progesterone receptor agonists or progesterone receptor modulators. Progestins bind to an intracellular receptor and mimic the action of the natural steroid hormone progesterone. Suitable nonsteroidal progestins for use in the present invention include, but are not limited to, natural and synthetic nonsteroidal compounds having a high affinity and specific selectivity for the progesterone receptor. An example of a nonsteroidal progestin includes, but is not limited to tanaproget, 5-(1,2-Dihydro-2-thioxospiro[cyclohexane-1,3-[3H]indol]-5-yl)-1-(tert-butoxycarbonyl)-pyrrole-2-carbonitrile, 5-(1,2-Dihydro-2-thioxospiro[cyclohexane-1,3-[3H]indol]-5-yl)-1-H-pyrrole-2-carbonitrile, 5-(2′-thioxospiro[cyclohexane-1,3′-[3H]indol]-5′-yl)-1-methyl-pyrrole-2-carbonitrile, 5-(1,2-Dihydro-2-thioxospiro[cyclopentane-1,3-[3H]indol]-5-yl)-3-thiophene carbonitrile, 5-(1,2-Dihydro-thioxospiro(cyclopentane-1,3-[3H]indol)-5-yl)-2-thiophenecarbonitrile, 5-(5-Chloro-2-thienyl)spiro[cyclohexane-1,3-[3H]indole]-2(1H)-thione, 5-(1,2-Dihydro-2-thioxospiro[cyclohexane-1,3-[3H]indol]-5-yl)-3-furancarbonitrile, 5-(1,2-Dihydro-2-thioxospiro[cyclohexane-1,3-[3H]indol]-5-yl)-4-propyl-2-thiophencarbonitrile, 4-(1,2-Dihydro-2-thioxospiro[cyclohexane-1,3-[3H]indol]-5-yl)-2-furancarbonitrile, 5-(1″,2″-Dihydro-2″-thioxospiro[cyclohexane-1,3″-[3H]indol]-5″-yl)-4-methyl-2-thiophenecarbonitrile, 5′-(1″,2″-Dihydro-2″-thioxospiro[cyclohexane-1,3″-[3H]indol]-5″-yl)-2-thiophenecarbonitrile, 5-(1,2-Dihydro-2-thioxospiro[cyclohexane-1,3-[3H]indol]-5-yl)-4-n-butyl-2-thiophenecarbonitrile, 5-(spiro[cyclohexane-1,3′-[3H]indol]-2′-(hydroxyimino)-5′-yl)-4-methyl-2-thiophenecarbonitrile, 5-(spiro[cyclohexane-1,3′-[3H]indole]-2′-(hydroxyimino)-5′-yl-2-thiophenecarbonitrile, 4-(Spiro[cyclohexane-1,3′-[3H]indole]-2′-(hydroxyimino)-5′-yl)-2-thiophene carbonitrile, 5-(spiro[cyclohexane-1,3′-[3H]indole]-2′-(hydroxyimino)-5′-yl)-1H-pyrrole-1-methyl-2-carbonitrile, 5-(spiro[cyclohexane-1,3′-[3H]indol]-2′-(hydroxyimino)-5′-yl)-1H-pyrrole-2-carbonitrile, 4-(spiro[cyclohexane-1,3′-[3H]indole]-2′-(acetoxyimino)-5′-yl)-2-thiophenecarbonitrile, N′-hydroxy-5-(spiro[cyclohexane-1,3′-[3H]indole]-2′-(hydroxyimino)-5′-yl)-4-methyl-2-thiophenecarboximidamide, N′-Hydroxy-4-(spiro[cyclohexane-1,3′-[3H]indole]-2′-(hydroxyimino)-5′-yl-2-thiophenecarboximidamide, N′-Hydroxy-5-(spiro[cyclohexane-1,3′-[3H]indol]-2′-(hydroxyimino)-5′-yl)-2-thiophenecarboximidamide, 5′-(5-Cyano-1H-pyrrol-2-yl)spiro[cyclohexane-1,3′-[3H]indol]-2′-ylidenecyanamide, 5′-(5-Cyano-thiophen-2-yl)spiro[cyclohexane-1,3′-[3H]indol]-2′-ylidenecyanamide, 5′-(5-Cyano-3-methyl-thiophen-2-yl)spiro[cyclohexane-1,3′-[3H]indol]-2′-ylidenecyanamide, 5′(5-Cyano-thiophene-3-yl)spiro[cyclohexane-1,3′-[3H]indol]-2′-ylidenecyanamide, 5-(spiro[cyclohexane-1,3′-[3H]indole]-2′-Cyanomethylene-5′-yl)-4-methylthiophene-2-carbonitrile, 4-(spiro[cyclohexane-1,3′-[3H]indole]-2′-Cyanomethylene-5′-yl)-thiophene-2-carbonitrile, (+/−)-5-{2-hydroxy-3-[1-(2-fluoro-5-trifluoromethylphenyl)-cyclopropyl]-2-trifluoromethyl-propionylamino}-phthalide ((±)-1), ((+)-1), and ((−)-1)); (+/−)-6-{2-hydroxy-3-[1-(2-fluoro-3-trifluoromethylphenyl)-cyclopropyl]-2-trifluoromethyl-propionylamino}-4-methyl-2,3-benzoxazin-1-one ((±)-2), ((+)-2), and ((−)-2)); (+/−)-6-{2-hydroxy-3-[1-(3-trifluoromethylphenyl)-cyclopropyl]-2-methyl-propionylamino}-4-methyl-2,3-benzoxazin-1-one ((±)-3), ((+)-3), and ((−)-3)); (+/−)-5-{2-hydroxy-3-[1-(2-fluoro-3-trifluoromethylphenyl)-cyclopropyl]-2-trifluoromethyl-propionylamino}-phthalide ((±)-4), ((+)-4), and ((−)-4)); (+/−)-6-{2-hydroxy-3-[1-(2-fluoro-5-trifluoromethylphenyl)-cyclopropyl]-2-trifluoromethyl-propionylamino}-4-methyl-2,3-benzoxazin-1-one ((±)-5), ((+)-5), and ((−)-5)); (+/−)-6-{2-hydroxy-3-[1-(2-fluoro-5-trifluoromethylphenyl)-cyclopropyl]-2-methyl-propionylamino}-4-methyl-2,3-benzoxazin-1-one ((±)-6), ((+)-6), and ((−)-6)); 5-aryl-1,2-dihydrochromomeno[3,4-j]quinolines. See U.S. Pat. No. 6,436,929; U.S. Pat. No. 6,355,648; U.S. Appl. Pub. No. 2003/0232824 A1; Fensome A. et al., J. Med. Chem. 48:5092-5095 (2005); Fensome A. et al., Bioorg. Med. Chem. Lett. 13(7): 1317-1320 (2003), and Zhi et al., J. Med. Chem., 41(3): 291-302 (1998). Each of these documents is hereby incorporated by reference in its entirety. Other chemical forms that provide a biologically active nonsteroidal progestin to female, including, for example, esters, conjugates, hydrates, prodrugs, solvates, and salts of suitable nonsteroidal progestins, can also be used.

As used herein, salts include, but are not limited to, acid addition salts such as, hydrochloric, hydrobromic, citric, tartaric, phosphoric, fumaric, malic, and succinic acids, sodium and potassium salts thereof, and combination thereof.

The expression “prodrug” denotes a derivative of a known direct acting drug, which derivative has enhanced delivery characteristics and therapeutic value as compared to the drug and is transformed into the active drug by an enzymatic or chemical process in the body of the female after administration.

In some embodiments, the nonsteroidal progestin used here is micronized. Micronized nonsteroidal progestin includes nonsteroidal progestin compositions in which the particles of the composition have been reduced to 20 microns or less in diameter. Particles size measuring devices are known in the art and include, e.g., devices produced by Malvern Instruments, Worcestershire, United Kingdom. Micronized nonsteroidal progestin is discussed, for example, in U.S. Appl. Pub. No. 2006/0247234 A1 and U.S. Appl. Pub. No. 2006/0246128 A1.

In some embodiments, the pharmaceutically effective amount of a nonsteroidal progestin for emergency contraception is about 2 times to about 1000 times the dosage required for daily contraception or non-emergency contraception. In some embodiments, the pharmaceutically effective amount of a nonsteroidal progestin for emergency contraception is about 10 times to about 40 times the average daily dose required for contraception. For example, a pharmaceutically effective amount of a nonsteroidal progestin for emergency contraception is equivalent to about 0.5 mg to about 20 mg tanaproget.

In some embodiments, the nonsteroidal progestin is tanaproget, although other suitable nonsteroidal progestins can be employed. In some embodiments, the pharmaceutically effective amount of tanaproget is in an amount of about 1 mg to about 20 mg. In some embodiments, the pharmaceutically effective amount of tanaproget is in an amount of about 3 mg to about 10 mg. For example, the method of the present invention can comprise administrating a dosage of tanaproget in the amount of 1 mg, 3 mg, 5 mg, 10 mg, 15 mg, or 20 mg in a single dosage.

The chemical name for tanaproget is [5-(4,4-Dimethyl-2-thioxo-1,4-dihydro-2H-3,1-benzoxazine-6-yl)-1-methyl-1H-pyrrole-2-carbonitrile]. Tanaproget can have the following chemical structure:

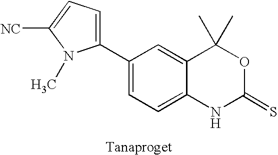

As used herein, “tanaproget” includes NSP-989 (CAS registration number: 304853-42-7), tanaproget Form I, Form II tanaproget polymorph, and its respective isomers, enantiomers, solvates, hydrates, and pharmaceutically acceptable salts thereof.

In some embodiments, the nonsteroidal progestin or tanaproget can be micronized. As used herein, micronized tanaproget means that the tanaproget particles have been reduced in size such that they are less than about 20 microns, less than about 15 μm, or less than about 10 μm in diameter. In some embodiments, 90% of the micronized particles are less than about 20 μm and 50% are less than about 15 μm or about 10 μm.

In some embodiments, tanaproget used in the dosage form can be purified. As used herein, “purified” means that tanaproget contains less than about 1% by weight impurities, about 0.5% by weight impurities, or about 0.36% by weight impurities. For example, a purified tanaproget can be about 99.5% by weight pure. In some embodiments, tanaproget can be purified by recrystallization.

In some embodiments, tanaproget used in the dosage form can be a Form II tanaproget polymorph. As used herein, the Form II tanaproget polymorph is prepared by recrystallizing non-micronized or micronized tanaproget Form I from selected solvent systems. The Form II tanaproget polymorph has a different differential scanning calorimetry thermogram and X-ray diffraction pattern from tanaproget Form I.

The compounds, compositions, pharmaceutical formulation, methods of making, using, and purifying tanaproget are described in U.S. Pat. No. 6,436,929; U.S. Pat. No. 7,081,457; U.S. Pat. No. 7,192,956; U.S. Appl. Pub. No. 2005/0250766 A1; U.S. Appl. Pub. No. 2005/0272702 A1; U.S. Appl. Pub. No. 2005/0239779 A1; U.S. Appl. Pub. No. 2006/0009428 A1; U.S. Appl. Pub. No. 2006/0074081 A1; U.S. Appl. Pub. No. 2006/0142280 A1; U.S. Appl. Pub. No. 2006/0246135 A1; U.S. Appl. Pub. No. 2006/0247234 A1; U.S. Appl. Pub. No. 2006/0247235 A1; U.S. Appl. Pub. No. 2006/0247236 A1; U.S. Appl. Pub. No. 2006/0246128 A1; U.S. Appl. Pub. No. 2006/0280800 A1; and U.S. Appl. Pub. No. 2007/0213526 A1. See also Bapst J. L., Contraception 74:414-418 (2006); and Zhang Z., J. Biol. Chem. 280(31):28468-28475 (2005). Each of these documents is hereby incorporated by reference in its entirety.

If a progestin different from tanaproget is employed, an adjustment in the amount administered can be made based on the relative potency or activity of the progestin relative to tanaproget. Equivalent concentrations of progestins relative to tanaproget can be determined using either in vitro or in vivo assay methods. See, for example, Kuhl, H., Drugs 51(2):188-215 (1996); Philibert, D., et al., Gynecol. Endocrinol. 13:316-326 (1999); and Lundeen, S., et al., J. Steroid Biochem. Molec. Biol. 78:137-143 (2001), in which the relative potencies of various progestins are compared using both in vitro and in vivo test assays. Each of these documents is hereby incorporated by reference in its entirety.

In some embodiments, a dosage of a nonsteroidal progestin further comprises a pharmaceutically effective amount of an estrogen. In some embodiments, this is an amount of estrogen equivalent to about 0.025 mg to about 0.4 mg of ethinyl estradiol. Correlations in potency among the various estrogens are known. See, for example, EP 0 253 607, which is incorporated herein in its entirety by reference. For example, 30 μg of ethinyl estradiol is roughly equivalent to 60 g of mestranol or 2,000 μg of 17β-estradiol. Equivalent concentrations of estrogens can be determined using either in vitro or in vivo assay methods.

Estrogens that can be used in the present invention include natural and synthetic estrogens, i.e., derived from natural and synthetic sources. Exemplary estrogens include estradiol, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17α-Δ^{8,9}-dehydroestradiol, 17β-Δ^{8,9}-dehydroestradiol, 6-OH-equilenin, 6-OH-17α-dihydroequilenin, 6-OH-17β-dihydroequilenin, estradiol benzoate, estradiol valerate, estrone, piperazine estrone sulfate, estriol, estriol succinate, polyestriol phosphate, ethinyl estradiol, 17α-ethinylestradiol, ethinyl estradiol-3 methyl ether, mestranol, quinestrol, quinestranol, conjugated equine estrogens, and mixtures, conjugates, and salts thereof, and the estrogen ketones and their corresponding 17α- and 17β-hydroxy derivatives. Other estrogens that can be used in the practice of the present invention include, e.g., the estrogenic compounds set forth in U.S. Pat. No. 6,660,726, and those set forth in U.S. Appl. Pub. No. 2003/0158432.

Prodrugs of estrogens can also be used in the method of the present invention. Examples of estrogen prodrugs that can be used in the present invention include, but are not limited to, estradiol acetate (which is converted in vivo to 17β-estradiol) and mestranol (which is converted in vivo to ethinyl estradiol).

Conjugated estrogens can be used in the methods of the invention. The conjugates can be various conjugates understood by those skilled in the art, including, but not limited to, sulfate and glucuronide conjugates. The estrogenic compounds can also be present as salts of estrogens conjugates. The salts can be various salts understood by those skilled in the art, including, but not limited to, sodium salts, calcium salts, magnesium salts, lithium salts, and piperazine salt.

The estrogen is administered substantially concurrent with the nonsteroidal progestin. As used herein, the term, “substantially concurrent” means that an estrogen can be administered 0.5 hr, 1 hr, 1.5 hrs, 2 hrs, 2.5 hrs, 3 hrs, 3.5 hrs, or 4 hrs after the administration of the nonsteroidal progestin. Alternatively, the estrogen can be administered at the same time as the nonsteroidal progestin, in the same or in a different dosage.

In some embodiments, the estrogen can be ethinyl estradiol. In some embodiments, the total pharmaceutically effective amount of ethinyl estradiol is about 0.025 mg to about 0.4 mg. For example, the method of the present invention can comprise a dosage of 1 mg, 3 mg, 5 mg, 10 mg, 15 mg, or 20 mg of tanaproget and a dosage of 0.05 mg, 0.1 mg, 0.15 mg, 0.2 mg, 0.25 mg, 0.3 mg, 0.35 mg, or 0.4 mg of ethinyl estradiol, in the same or different dosages.

In some embodiments, a pharmaceutically effective amount of a nonsteroidal progestin is in a single dosage. For example, a single tablet, pill, or capsule comprising a nonsteroidal progestin equivalent to about 0.5 mg to about 20 mg tanoproget is administered. A single dosage would also include multiple dosage forms (e.g., tablets, pills, capsules, etc.) which are administered closely together in time, i.e., within 30 minutes, 10 minutes, 5 minutes, or 2 minutes of each other, the sum of the amount of the nonsteroidal progestin of the multiple dosage forms being equivalent to about 0.5 mg to about 20 mg tanoproget. In some embodiments, a pharmaceutically effective amount of a nonsteroidal progestin in a first dosage effective for emergency contraception further comprises a second dosage of a pharmaceutically effective amount of a nonsteroidal progestin.

In some embodiments, a second dosage comprising a nonsteroidal progestin equivalent to about 0.5 mg to about 10 mg of tanaproget is administered.

In some embodiments, the nonsteroidal progestin of a first dosage is administered to a female in need of emergency contraception, and then a second dosage is administered to the female, within about 96 hours postcoitus (or after intercourse or undesired insemination), within about 72 hours postcoitus, within about 48 hours postcoitus, or within about 24 hours postcoitus. For example, the nonsteroidal progestin of a first dosage is administered to a female in need of emergency contraception, and a second dosage is administered to the same female within about 12 hours, within about 24 hours, or within about 36 hours after administration of the first dosage, and all the dosages are administered within about 96 hours postcoitus, within about 72 hours postcoitus, within about 48 hours postcoitus, or within about 24 hours postcoitus.

In some embodiments, the nonsteroidal progestin in both the first and the second dosages is tanaproget.

In some embodiments, the tanaproget in each of the first and the second dosages is in an amount of about 0.5 mg to about 10 mg. In some embodiments, the tanaproget in each of the first and the second dosages is in an amount of about 1.5 mg to about 5 mg. For example, the method of the present invention can comprise two dosages of tanaproget in the amount of 0.5 mg, 1.5 mg, 2.5 mg, 5 mg, 7.5 mg, or 10 mg, wherein the second dosage is administered to the female within about 4, 12, 20, 28, or 36 hours after administration of the first dosage, and both dosages are administered to the female within about 72 hours postcoitus.

In some embodiments, the first and/or the second dosage further comprise a pharmaceutically effective amount of an estrogen. In some embodiments, the second dosage further comprises an estrogen equivalent to about 0.025 mg to about 0.4 mg of ethinyl estradiol.

In some embodiments, the method of the present invention can comprise two dosages: the first dosage comprising 0.5 mg, 1.5 mg, 2.5 mg, 5 mg, 7.5 mg, or 10 mg of tanaproget and the second dosage comprising 0.025 mg, 0.05 mg, 0.075 mg, 0.1 mg, 0.125 mg, 0.15 mg, 0.175 mg, or 0.2 mg of ethinyl estradiol, wherein the second dosage is administered within about 4, about 12, about 20, about 28, or about 36 hours of administration of the first dosage, and wherein both the first dosage and the second dosage are administered within about 96 hours postcoitus or within about 72 hours postcoitus.

In some embodiments, the method of the present invention can comprise two dosages: the first dosage comprising 0.5 mg, 1.5 mg, 2.5 mg, 5 mg, 7.5 mg, or 10 mg of tanaproget and 0.025 mg, 0.05 mg, 0.075 mg, 0.1 mg, 0.125 mg, 0.15 mg, 0.175 mg, or 0.2 mg of ethinyl estradiol, and the second dosage also comprising 0.5 mg, 1.5 mg, 2.5 mg, 5 mg, 7.5 mg, or 10 mg of tanaproget and 0.025 mg, 0.05 mg, 0.075 mg, 0.1 mg, 0.125 mg, 0.15 mg, 0.175 mg, or 0.2 mg of ethinyl estradiol, wherein the second dosage is administered within about 4, about 12, about 20, about 28, or about 36 hours of administration of the first dosage, and wherein both the first dosage and the second dosage are administered within about 96 hours postcoitus or within about 72 hours postcoitus.

In some embodiments, the method of the present invention can comprise two dosages: the first dosage comprising 0.5 mg, 1.5 mg, 2.5 mg, 5 mg, 7.5 mg, or 10 mg of tanaproget, and the second dosage also comprising 0.5 mg, 1.5 mg, 2.5 mg, 5 mg, 7.5 mg, or 10 mg of tanaproget, wherein the second dosage is administered within about 4, about 12, about 20, about 28, or about 36 hours of administration of the first dosage, and wherein both the first dosage and the second dosage are administered within about 96 hours postcoitus or within about 72 hours postcoitus.

In some embodiments, the method of the present invention can comprise two dosages: the first dosage comprising 0.5 mg, 1.5 mg, 2.5 mg, 5 mg, 7.5 mg, or 10 mg of tanaproget and 0.025 mg, 0.05 mg, 0.075 mg, 0.1 mg, 0.125 mg, 0.15 mg, 0.175 mg, or 0.2 mg of ethinyl estradiol, and the second dosage comprising 0.5 mg, 1.5 mg, 2.5 mg, 5 mg, 7.5 mg, or 10 mg of tanaproget, wherein the second dosage is administered within about 4, about 12, about 20, about 28, or about 36 hours of administration of the first dosage, and wherein both the first dosage and the second dosage are administered within about 96 hours postcoitus or within about 72 hours postcoitus.

In some embodiments, the method of the present invention can comprise two dosages: the first dosage comprising 0.5 mg, 1.5 mg, 2.5 mg, 5 mg, 7.5 mg, or 10 mg of tanaproget and 0.025 mg, 0.05 mg, 0.075 mg, 0.1 mg, 0.125 mg, 0.15 mg, 0.175 mg, or 0.2 mg of ethinyl estradiol, and the second dosage comprising 0.025 mg, 0.05 mg, 0.075 mg, 0.1 mg, 0.125 mg, 0.15 mg, 0.175 mg, or 0.2 mg of ethinyl estradiol, wherein the second dosage is administered within about 4, about 12, about 20, about 28, or about 36 hours of administration of the first dosage, and wherein both the first dosage and the second dosage are administered within about 96 hours postcoitus or within about 72 hours postcoitus.

The nonsteroidal progestin and any other desired additional active pharmaceutical (such as estrogen) are administered in the conventional manner by any route that retains, as provided far, the bioactivity of the desired agent. For example, administration can be by, but is not limited to, administration by methods that are parenterally, subcutaneously, intravenously, intramuscularly, transdermally, buccally, orally, or intravaginally. Thus, the dosage forms comprising nonsteroidal progestin and with any other desired additional active pharmaceutical (such as estrogen) can be, but are not limited to, sublingual, injectable (including short-acting, pellet forms injected subcutaneously or intramuscularly), vaginal creams, suppositories, pessaries, rings, rectal suppositories, and transdermal forms, such as patches and creams.

In some embodiments, when there are two or more active pharmaceutical components, if desired, the different active components may be administered to the female by the same or different routes or forms of administration. For example, for a regimen that requires both the nonsteroidal progestin and the estrogen, the estrogen can be provided by transdermal administration, and the nonsteroidal progestin can be provided by vaginal administration. As another example, the estrogen can be provided by transdermal administration, and the nonsteroidal progestin can be provided by oral administration. As another example, both the estrogen and the nonsteroidal progestin can be provided by oral administration.

The nonsteroidal progestin, any other desired additional active pharmaceutical (such as estrogen), and a suitable carrier can be in solid dosage forms which include, but are not limited to, tablets, capsules, cachets, pellets, pills, powders, and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder. It is known in the art that the active ingredients can be contained in such compositions with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, “Modern Pharmaceutics”, Banker & Rhodes, Marcel Dekker, Inc. 1979; and “Goodman & Gilman's The Pharmaceutical Basis of Therapeutics,” 6^{th} Edition, MacMillan Publishing Co., New York 1980 can be consulted.

In some embodiments, the nonsteroidal progestin and any other desired additional active pharmaceutical (such as estrogen) can be formulated readily by combining these compounds with pharmaceutically acceptable carriers well known in the art. Such carriers facilitate formulating the nonsteroidal progestins of the invention as tablets, pills, dragees, capsules, liquids, gels, syrups, slurries, suspensions and the like, for oral ingestion by a patient to be treated. Pharmaceutical preparations for oral administration can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP).

If desired, disintegrating agents can be added, such as, but not limited to, the cross-linked polyvinyl pyrrolidone, agar, or alginic acid or a salt thereof such as sodium alginate. For example, the nonsteroidal progestin and any other desired additional active pharmaceutical (such as estrogen) can be formulated for orally disintegrating tablets. Orally disintegrating tablets (ODTs) are known in the art. See, e.g., U.S. Pat. Nos. 6,368,625 and 6,316,029, each of which is hereby incorporated by reference in its entirety.

The nonsteroidal progestin and any other desired additional active pharmaceutical (such as estrogen) also can be formulated comprising suitable solid or gel phase carriers or excipients such as calcium carbonate, calcium phosphate, various sugars, starches, cellulose derivatives, gelatin, and polymers such as, e.g., polyethylene glycols. Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All compositions for oral administration should be in dosages suitable for such administration. For example, the nonsteroidal progestin and any other desired additional active pharmaceutical (such as estrogen) can be formulated for chewable tablets. Chewable tablets are known in the art. See, e.g., U.S. Pat. Nos. 4,684,534 and 6,060,078, each of which is incorporated by reference in its entirety.

Buccal administration of a composition that comprises the nonsteroidal progestin and any other desired additional active pharmaceutical (such as an estrogen) can take the form of tablets or lozenges formulated in conventional manner.

The compounds can also be formulated in rectal compositions such as suppositories or retention enemas, e.g., containing conventional suppository bases such as cocoa butter or other glycerides.

Transdermal administration of a composition that comprises the nonsteroidal progestin and any other desired additional active pharmaceutical (such as an estrogen) can be applied to a plaster or a transdermal patches, both of which are known in the art, for delivery across the skin of a female. Such nonsteroidal progestin containing composition can also be transdermal therapeutic systems or devices. Devices or systems known to the art include reservoir type devices involving membranes that control the rate of drug release to the skin and devices involving a dispersion of the drug in a matrix.

In some embodiments, the composition that comprises the nonsteroidal progestin and any other desired additional active pharmaceutical (such as estrogen) can be applied to a transdermal patch. The transdermal patch can release a dosage of nonsteroidal progestin and any other desired additional active pharmaceutical (such as an estrogen) within about 2 hours, within about 12 hours, within about 24 hours, within about 36 hours, within about 48 hours, within about 72 hours, or within about 96 hours postcoitus.

In some embodiments, the nonsteroidal progestin and any other desired additional active pharmaceutical (such as estrogen) are in an oral, transdermal, intravaginal, or injectable liquid dosage form.

For transvaginal administration, a transvaginal ring can be used to release a composition that comprises the nonsteroidal progestin and any other desired additional active pharmaceutical (such as an estrogen) in a pharmaceutically effective amount. In some embodiments, a transvaginal ring can release such composition in a pharmaceutically effective amount within about 2 hours, within about 12 hours, within about 24 hours, within about 36 hours, within about 48 hours, within about 72 hours, or within about 96 hours postcoitus.

### Pharmaceutical Packages

The present invention is directed to a pharmaceutical package for emergency contraception, the package comprising:
(a) a dosage comprising nonsteroidal progestin in a pharmaceutically effective amount for emergency contraception;
(b) a suitable container; and
(c) a label directing administering the nonsteroidal progestin to a female in need thereof within about 96 hours postcoitus.

The pharmaceutical packages of the present invention are designed for use in the regimens described herein.

In some embodiments, the nonsteroidal progestin in the package is tanaproget. In some embodiments, the tanaproget in the package is in a dosage of an amount of about 1 mg to about 20 mg. In other embodiments, the tanaproget in the package is a dosage of an amount of about 3 mg to about 10 mg.

In some embodiments, the nonsteroidal progestin in the package comprises two dosages, each dosage comprising a pharmaceutically effective amount of a nonsteroidal progestin.

As used herein, the “suitable container” is used for storing each component of the package. The container can be, for example, a bag, box, envelope or any other container that would be suitable for use in the present invention. The container can be large enough to accommodate each component and/or any administrative devices that may be necessary for use of the dosage of the package according to the methods of the present invention. A container can also be small enough to fit into a pocketbook, briefcase, or pocket.

In some embodiments, a suitable container is a blister pack, containing a single dosage or two dosages of a nonsteroidal progestin and any other desired additional active pharmaceutical (such as estrogen).

As used herein, “a label directing administering the nonsteroidal progestin” refers to instructions associated with the dosage of the package. Such instructions can be in a form prescribed by a governmental agency regulating the manufacture, use or sale of pharmaceuticals or biological products, which notice reflects approval by the agency of the manufacture, use or sale for human administration to treat a condition or disorder using the methods or regimens described herein. The instructions can be in any form which conveys information on the use of the dosage units in the kit according to the methods of the invention. For example, the instructions can be in the form of printed matter, or in the form of a pre-recorded media device.

“Printed matter” can be, for example, one of a book, booklet, brochure or leaflet. The printed matter can describe the use of the dosage according to the methods of the present invention. Possible formats include, but are not limited to, a bullet point list, a list of frequently asked questions (FAQ) or a chart. Additionally, the information to be imparted can be illustrated in non-textual terms using pictures, graphics or other symbols.

“Pre-recorded media device” can be, for example, a visual media device, such as a videotape cassette, a DVD (digital video disk), filmstrip, 35 mm movie or any other visual media device. Alternatively, pre-recorded media device can be an interactive software application, such as a CD-ROM (compact disk-read only memory) or floppy disk. Alternatively, pre-recorded media device can be, for example, an audio media device, such as a record, audiocassette or audio compact disk. The information contained on the pre-recorded media device can describe the proper use of the dosage according to the methods of the present invention, e.g., for emergency contraception.

In some embodiments, the label of the package directs administering the nonsteroidal progestin immediately, as soon as possible, within about 96 hours, within about 72 hours, within about 48 hours, within about 24 hours, or within about 12 hours postcoitus to a female in need of emergency contraception.

In some embodiments, the nonsteroidal progestin and any other desired additional active pharmaceutical (such as estrogen) of the package of the present invention are designed for oral, transdermal, transvaginal, or injectable liquid dosage form, as described for use in the regimens described herein.

In some embodiments, the nonsteroidal progestin and any other desired additional active pharmaceutical (such as estrogen) of the packages of the present invention can be in solid dosage forms which include, but are not limited to, tablets, capsules, cachets, pellets, pills, powders, and granules.

The following examples are illustrative, but not limiting, of the method and compositions of the present invention. Other suitable modifications and adaptation of the variety of conditions and parameters normally encountered and obvious to those skilled in the art are within the spirit and scope of the invention. Thus, the breadth and scope of the present invention should not be limited by any of the above-described exemplary embodiments, but should be defined only in accordance with the following claims and their equivalents.

### EXAMPLES

### Example 1

Table 1 below shows examples of several regimens comprising nonsteroidal progestin for emergency contraception. Each regimen comprises administration of tanaproget in a single or two dosages. It is anticipated that these regimens will proved similar efficacy comparing to the known ECP regimens, i.e., progestin-only regimen, combined or Yuzpe regimen, or mifepristone method.

Regimens

| | |
|---|---|
| A | Two dosages of 1.5 mg of tanaproget 12 hours apart, |
| | both administered within about 72 hours postcoitus. |
| B | Two dosages of 2.5 mg of tanaproget 12 hours apart, |
| | both administered within about 72 hours postcoitus. |
| C | Two dosages of 5 mg of tanaproget 12 hours apart, |
| | both administered within about 72 hours postcoitus. |
| D | Single dosage of 3 mg of tanaproget administered |
| | within about 72 hours postcoitus. |
| E | Single dosage of 5 mg of tanaproget administered |
| | within about 72 hours postcoitus. |
| F | Single dosage of 10 mg of tanaproget administered |
| | within about 72 hours postcoitus. |
| G | Two dosages, each comprising 1.5 mg tanaproget and |
| | 0.025 mg ethinyl estradiol 12 hours apart, both |
| | administered within about 72 hours postcoitus. |
| H | Two dosages, each comprising 3 mg tanaproget and |
| | 0.05 mg ethinyl estradiol 12 hours apart, both |
| | administered within about 72 hours postcoitus. |
| I | Two dosages, each comprising 5 mg tanaproget and 0.2 |
| | mg ethinyl estradiol 12 hours apart, both administered |
| | within about 72 hours postcoitus. |
| J | Single dosage of 3 mg tanaproget and 0.05 mg ethinyl |
| | estradiol administered within about 72 hours postcoitus. |
| K | Single dosage of 6 mg tanaproget and 0.1 mg ethinyl |
| | estradiol administered within about 72 hours postcoitus. |
| L | Single dosage of 10 mg tanaproget and 0.4 mg ethinyl |
| | estradiol administered within about 72 hours postcoitus. |

While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All of the various embodiments or options described herein can be combined in any and all variations. All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. A method for emergency contraception, comprising administering to a female in need thereof a dosage comprising a nonsteroidal progestin equivalent to about 0.5 mg to about 20 mg of tanaproget, wherein the dosage is effective for emergency contraception.

2. The method of claim 1, wherein the nonsteroidal progestin is administered within about 72 hours postcoitus.

3. The method of claim 1, wherein the nonsteroidal progestin is administered orally.

4. The method of claim 1, wherein the nonsteroidal progestin is tanaproget.

5. The method of claim 4, wherein the tanaproget is in an amount of about 1 mg to about 20 mg.

6. The method of claim 4, wherein the tanaproget is an amount of about 3 mg to about 10 mg.

7. The method of claim 1, wherein the dosage further comprises an estrogen equivalent to about 0.025 mg to about 0.4 mg of ethinyl estradiol.

8. The method of claim 7, wherein the estrogen is ethinyl estradiol.

9. The method of claim 1, further comprising administering a second dosage comprising a nonsteroidal progestin equivalent to about 0.5 mg to about 10 mg of tanaproget to the female in need thereof.

10. The method of claim 9, wherein the second dosage is administered within about 36 hours after administration of a first dosage.

11. The method of claim 9, wherein the second dosage is administered within about 12 hours after administration of a first dosage.

12. The method of claim 9, wherein the nonsteroidal progestin is tanaproget.

13. The method of claim 12, wherein the tanaproget in a first and the second dosages is in an amount of about 0.5 mg to about 10 mg.

14. The method of claim 12, wherein the tanaproget in a first and the second dosages is in an amount of about 1.5 mg to about 5 mg.

15. The method of claim 9, wherein a first or the second dosage further comprises an estrogen equivalent to about 0.025 mg to about 0.4 mg of ethinyl estradiol.

16. The method of claim 15, wherein the estrogen is ethinyl estradiol.

17. A method for emergency contraception, comprising administering within about 96 hours postcoitus to a female in need thereof a pharmaceutically effective amount of a nonsteroidal progestin for emergency contraception.

18. The method of claim 17, wherein the nonsteroidal progestin is administered within about 72 hours postcoitus.

19. The method of claim 17, wherein the nonsteroidal progestin is administered within about 48 hours postcoitus.

20. The method of claim 17, wherein the nonsteroidal progestin is administered within about 24 hours postcoitus.

21. The method of claim 17, wherein the nonsteroidal progestin is administered orally.

22. The method of claim 17, wherein the nonsteroidal progestin is tanaproget.

23. The method of claim 17, further comprising administering a pharmaceutically effective amount of an estrogen substantially concurrent with the nonsteroidal progestin.

24. The method of claim 23, wherein the estrogen is ethinyl estradiol.

25. (canceled)

26. (canceled)

27. A pharmaceutical package for emergency contraception, the package comprising:
(a) a dosage comprising a nonsteroidal progestin in a pharmaceutically effective amount for emergency contraception;
(b) a suitable container; and
(c) a label directing administering the nonsteroidal progestin to a female in need thereof within about 96 hours postcoitus.

28. The package of claim 27, wherein the nonsteroidal progestin is tanaproget.

29. The package of claim 28, wherein the tanaproget is in an amount of about 1 mg to about 20 mg.

30. The package of claim 28, wherein the tanaproget is in an amount of about 3 mg to about 10 mg.

31. The package of claim 27, wherein the dosage is in a form of a capsule or a tablet.

32. The package of claim 27, wherein the label directs administering the nonsteroidal progestin within about 72 hours postcoitus to the female in need thereof.

33. The package of claim 27, comprising two dosages comprising a nonsteroidal progestin.

34. The package of claim 27, further comprising a dosage comprising an estrogen equivalent to about 0.025 mg to about 0.4 mg of ethinyl estradiol.

35. The package of claim 34, wherein the estrogen is ethinyl estradiol.

36. A pharmaceutical composition comprising a nonsteroidal progestin in a pharmaceutically effective amount for emergency contraception.

37. The composition of claim 36, wherein the nonsteroidal progestin is tanaproget.

38. The composition of claim 37, wherein the tanaproget is in an amount of about 1 mg to about 20 mg.

39. The composition of claim 37, wherein the tanaproget is in an amount of about 3 mg to about 10 mg.

40. The composition of claim 37, wherein the composition is in a form of a capsule or a tablet.

41. The composition of claim 37, further comprising an estrogen equivalent to about 0.025 mg to about 0.4 mg of ethinyl estradiol.

42. The composition of claim 41, wherein the estrogen is ethinyl estradiol.
